(19) United States
(12) Patent Application Publication
(10) Pub. No.: US 2014/0357878 A1
(43) Pub. Date: Dec. 4, 2014

(54) METHOD OF PRODUCING 5-HYDROXYMETHYLFURFURAL FROM CARBOHYDRATES

(71) Applicant: AGENCY FOR SCIENCE , TECHNOLOGY AND RESEARCH, Singapore (SG)

(72) Inventors: Yugen Zhang, Singapore (SG); Linke Lai, Singapore (SG)

(73) Assignee: Agency for Science, Technology and Research, Singapore (SG)

(21) Appl. No.: 14/346,678

(22) PCT Filed: Sep. 24, 2012
(86) PCT No.: PCT/SG2012/000353

§ 371 Date: Mar. 21, 2014

(30) Foreign Application Priority Data
Sep. 23, 2011 (SG) 201106913-5

(51) Int. Cl. C07D 307/48

(52) U.S. Cl. CPC C07D 307/48
USPC 549/488

(57) ABSTRACT

Disclosed herein is a process for preparing 5-hydroxymethylfurfural comprising the step of contacting a carbohydrate and a Brønsted acid in an alcoholic solvent comprising an alcohol selected from the group consisting of secondary alcohols, tertiary alcohols, aryl alcohols and combinations thereof under conditions to dehydrate the carbohydrate thereby forming a reaction product containing 5-hydroxymethylfurfural.

### TECHNICAL FIELD

The present invention generally relates to a process for producing 5-hydroxymethylfurfural from carbohydrates.

### BACKGROUND

The primary carbon feedstock for a wide variety of commodity and specialty chemicals, as well as for thermal and energy transportation, remains based on the fossil-fuel reservoir. However, diminishing hydrocarbon reserves has raised concerns over its scarcity in the decades to follow. The finite reserves of hydrocarbon-based fuels therefore lead to a demand for the development of a renewable resource. In this regard, biomass-derived carbohydrates represent a promising carbon-based, alternative energy source and a sustainable chemical feedstock.

Significant research has been undertaken with respect to providing processes for converting biomass to 5-hydroxymethylfurfural (“5-HMF”) and/or its derivatives. 5-HMF is a versatile and key intermediate in biofuel chemistry and thus possesses industrial utility, particularly in the petrochemical industry. However, 5-HMF has not seen widespread industrial use due to its prohibitively high production cost and other challenges associated with producing HMF, including the consumption of vast amounts of organic solvents, and with it, the attendant environmental costs necessitated by the disposal of such solvents.

Furthermore, the current low conversion yields chronically present in known synthesis processes also lead to wastage of raw materials in order to achieve an economically viable level of 5-HMF output. Additionally, the high solubility of 5-HMF in water generates further difficulties in 5-HMF production processes, especially with regard to isolation and purification steps. In currently known methods for 5-HMF synthesis, 5-HMF is usually obtained in a polar high boiling point solution. In these methods, it is necessary to provide efficient separation steps in order to make 5-HMF synthesis economically viable for industrial-scale production. Typically, reaction solvents used in the HMF synthesis include water, DMSO (dimethyl sulfoxide) or DMF (dimethylformamide), ionic liquids or a mixture thereof. HMF can be extracted using various organic solvents, such as MIBK (methyl isobutyl ketone), DCM (dichloromethane), ethyl acetate, THF (tetrahydrofuran), diethyl ether, or acetone. However, due to the high polarity of HMF, the isolation step typically requires multiple runs of a solvent-intensive, liquid-liquid extraction process.

Ionic liquid-organic solvent biphasic systems have been proposed to overcome the above extraction problem. However, biphasic extraction systems inevitably require the use of large amounts of organic solvents, which is both costly and poses disposal problems. Yet a further challenge in using biphasic extraction systems resides in the need to recycle the reaction media, such as the ionic liquids and catalysts, which in turn require more complex reactor designs and increases the overall production costs.

Accordingly, there is a need to provide a process for the production of 5-HMF that overcomes or at least ameliorates the above described technical problems.

### SUMMARY

According to a first aspect, there is provided a process for preparing 5-hydroxymethylfurfural comprising the step of contacting a carbohydrate and a. Brønsted acid in an alcoholic solvent comprising an alcohol selected from the group consisting of secondary alcohols, tertiary alcohols, aryl alcohols, and combinations thereof under conditions to dehydrate the carbohydrate thereby forming a reaction product containing 5-hydroxymethylfurfural.

Advantageously, as will be further described below, the provision of an alcoholic solvent comprising an alcohol selected from the group consisting of secondary alcohols, tertiary alcohols, aryl alcohols and combinations thereof, has been found to lead to unexpectedly high yields (up to 85%) of 5-hydroxymethylfurfural. Also importantly, it has also been found that the provision of the defined alcoholic solvents leads to high selectivity of 5-hydroxymethylfurfural and substantially prevents the formation of less desirable alkoxylated side-products. In some embodiments of the disclosed process, a selectivity of about 100% 5-HMF is achieved, i.e., the reaction product consists essentially of 5-hydroxymethylfurfural.

Further advantageously, the disclosed process avoids the use of large amounts of organic solvent and as such minimizes any attendant environmental impact. Accordingly, the disclosed process provides a simple process for HMF production and isolation, and is capable of scaling up for industrial output while maintaining economic feasibility.

### DEFINITIONS

The following words and terms used herein shall have the meaning indicated:

As used herein, the term “alkyl group” includes within its meaning monovalent (“alkyl”) and divalent (“alkylene”) straight chain or branched chain saturated aliphatic groups having from 1 to 10 carbon atoms, e.g., 1, 2, 3, 4, 5, 6, 7, 8, 9, or 10 carbon atoms. For example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, and the like.

The term “heteroalkyl” refers to a straight-or branched-chain alkyl group having from 2 to 12 atoms in the chain, one or more of which is a heteroatom selected from S, O, and N. Exemplary heteroalkyls include alkyl ethers, secondary and tertiary alkyl amines, alkyl sulfides, and the like.

The term “alkenyl group” includes within its meaning monovalent (“alkenyl”) and divalent (“alkenylene”) straight or branched chain unsaturated aliphatic hydrocarbon groups having from 2 to 10 carbon atoms, e.g., 2, 3, 4, 5, 6, 7, 8, 9, or 10 carbon atoms and having at least one double bond, of either. E, Z, cis or trans stereochemistry where applicable, anywhere in the alkyl chain. Examples of alkenyl groups include but are not limited to ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like.

The term “alkynyl group” as used herein includes within its meaning monovalent (“alkynyl”) and divalent (“alkynylene”) straight or branched chain unsaturated aliphatic hydrocarbon groups having from 2 to 10 carbon atoms and having at least one triple bond anywhere in the carbon chain. Examples of alkynyl groups include but are not limited to ethynyl, 1-propynyl, 1-butynyl, 2-butynyl, 1-methyl-2-butynyl, 3-methyl-1-butynyl, 1-pentynyl, 1-hexynyl, methylpentynyl, 1-heptynyl, 2-heptynyl, 1-octynyl, 2-octynyl, 1-nonyl, 1-decynyl, and the like.

The term “cycloalkyl” as used herein refers to cyclic saturated aliphatic groups and includes within its meaning monovalent (“cycloalkyl”), and divalent (“cycloalkylene”), saturated, monocyclic, bicyclic, polycyclic or fused polycyclic hydrocarbon radicals having from 3 to 10 carbon atoms, eg, 3, 4, 5, 6, 7, 8, 9, or 10 carbon atoms. Examples of cycloalkyl groups include but are not limited to cyclopropyl, 2-methylcyclopropyl, cyclobutyl, cyclopentyl, 2-methylcyclopentyl, 3-methylcyclopentyl, cyclohexyl, and the like.

The term “cycloalkenyl” as used herein, refers to cyclic unsaturated aliphatic groups and includes within its meaning monovalent (“cycloalkenyl”) and divalent (“cycloalkenylene”), monocyclic, bicyclic, polycyclic or fused polycyclic hydrocarbon radicals having from 3 to 10 carbon atoms and having at least one double bond, of either E, Z, cis or trans stereochemistry where applicable, anywhere in the alkyl chain. Examples of cycloalkenyl groups include but are not limited to cyclopropenyl, cyclopentenyl, cyclohexenyl, and the like.

The term “heterocycloalkyl” as used herein, includes within its meaning monovalent (“heterocycloalkyl”) and divalent (“heterocycloalkylene”), saturated, monocyclic, bicyclic, polycyclic or fused hydrocarbon radicals having from 3 to 10 ring atoms wherein 1 to 5 ring atoms are heteroatoms selected from O, N, NH, or S. Examples include azetidinyl, oxiranyl, cyclohexylimino, imdazolidinyl, imidazolinyl, morpholinyl, piperazinyl, piperidinyl, pyridyl, pyrazolidinyl, pyrazolinyl, pyrrolidinyl, pyrrolinyl, quinuclidinyl, tetrahydrofuranyl, tetrahydrothiophenyl, tetrahydropyranyl, and the like.

The term “heterocycloalkenyl” as used herein, includes within its, meaning monovalent (“heterocycloalkenyl”) and divalent (“heterocycloalkenylene”), saturated, monocyclic, bicyclic, polycyclic or fused polycyclic hydrocarbon radicals having from 3 to 10 ring atoms and having at least double bond, wherein from 1 to 5 ring atoms are heteroatoms selected from O, N, NH or S.

The term “heteroaromatic group” and variants such as “heteroaryl” or “heteroarylene” as used herein, includes within its meaning monovalent (“heteroaryl”) and divalent (“heteroarylene”), single, polynuclear, conjugated and fused aromatic radicals having 6 to 20 atoms wherein 1 to 6 atoms are heteroatoms selected from O, N, NH and S. Examples of such groups include benzimidazolyl, benzisoxazolyl, benzofuranyl, benzopyrazolyl, benzothiadiazolyl, benzothiazolyl, benzothienyl, benzotriazolyl, benzoxazolyl, furanyl, furazanyl, furyl, imidazolyl, indazolyl, indolizinyl, indolinyl, indolyl, isobenzofuranyl, isoindolyl, isothiazolyl, isoxazolyl, oxazolyl, phenanthrolinyl, purinyl, pyrazinyl, pyrazolyl, pyridazinyl, pyridinyl, 2,2′-pyridinyl, pyrimidinyl, pyrrolyl, quinolinyl, quinolyl, thiadiazolyl, thiazolyl, thiophenyl, triazolyl, and the like.

The term “halogen” or variants such as “halide” or “halo” as used herein refers to fluorine, chlorine, bromine and iodine.

The term “heteroatom” or variants such as “hetero-” as used herein refers to O, N, NH and S.

The term “alkoxy” as used herein refers to straight chain or branched alkyloxy groups. Examples include methoxy, ethoxy, n-propoxy, isopropoxy, tert-butoxy, and the like.

The term “amino” as used herein refers to groups of the form —NRaRb wherein Ra and Rb are individually selected from the group including but not limited to hydrogen, optionally substituted alkyl, optionally substituted alkenyl, optionally substituted alkynyl, and optionally substituted, aryl groups.

The term “aromatic group”, or variants such as “aryl” or “arylene” as used herein refers to monovalent (“aryl”) and divalent (“arylene”) single, polynuclear, conjugated and fused residues of aromatic, hydrocarbons having from 6 to 10 carbon atoms. Examples of such groups include phenyl, biphenyl, naphthyl, phenanthrenyl, and the like.

The term “aralkyl” as used herein, includes within its meaning monovalent (“aryl”) and divalent (“arylene”), single, polynuclear, conjugated and fused aromatic hydrocarbon radicals attached to divalent, saturated, straight and branched chain alkylene radicals.

The term “heteroaralkyl” as used herein, includes within its meaning monovalent (“heteroaryl”) and divalent (“heteroarylene”), single, polynuclear, conjugated and fused aromatic, hydrocarbon radicals attached to divalent saturated, straight and branched chain alkylene radicals.

The term “optionally substituted” as used herein means the group to which this term refers may be unsubstituted, or may be substituted with one or more groups independently selected from alkyl, alkenyl, alkynyl, thioalkyl, cycloalkyl, cycloalkenyl, heterocycloalkyl, halo, carboxyl, haloalkyl, haloalkynyl, hydroxyl, alkoxy, thioalkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocycloxy, heterocycloamino, haloheterocycloalkyl, alkylsulfenyl, alkylcarbonyloxy, alkylthio, acylthio, phosphorus-containing groups such as phosphono and phosphinyl, aryl, heteroaryl, alkylaryl, alkylheteroaryl, cyano, cyanate, isocyanate, —C(O)NH(alkyl), and —C(O)N(alkyl)2.

The term “haloalkyl” refers to a straight-or branched-chain alkenyl group having from 2-12 carbon atoms in the chain and where one or more hydrogens is substituted with a halogen. Illustrative haloalkyl groups include trifluoromethyl, 2-bromopropyl, 3-chlorohexyl, 1-iodo-isobutyl, and the like.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more, typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DISCLOSURE OF OPTIONAL EMBODIMENTS

Exemplary, non-limiting embodiments of the process according to the first aspect will now be disclosed.

According to a first aspect, there is provided a process for preparing 5-hydroxymethylfurfural comprising the step of contacting a carbohydrate and a Brønsted acid in an alcoholic solvent comprising an alcohol selected from the group consisting of secondary alcohols, tertiary alcohols, aryl alcohols, and combinations thereof under conditions to dehydrate the carbohydrate thereby forming a reaction product containing 5-hydroxymethylfurfural.

In certain embodiments, the alcohol is selected from the group consisting of secondary alcohols, tertiary alcohols, and combinations thereof.

Suitable carbohydrates useful in connection with the process include hexoses, such as glucose and fructose, cellulose, starch, glycogen, and sources of fructose, sources of glucose, and combinations thereof.

Sources of fructose can include fructose itself, purified or crude, or any biomass that contains fructose or precursors to fructose, such as corn syrup, sucrose, and polyfructose.

Sources of glucose can include glucose itself, purified or crude, or any biomass that contains glucose or precursors to glucose, such as corn syrup, sucrose, and polyglucose.

Hexoses are monosacharides having six carbon atoms and can be represented by the formula C₆H₁₂O₆. Suitable hexoses include aldohexoses and ketohexoses. The hexose may be present in acyclic form, cyclic hemiacetal form or hemiketal form, and combinations thereof. Any hexose stereoisomer can be used in connection with the processes disclosed herein, including naturally occurring hexoses, synthetic hexoses, and semi-synthetic hexoses. Particularly useful hexoses include, but are not limited to D-allose D-altrose, D-glucose, D-mannose, D-gulose, D-idose, D-galactose, D-talose, D-psicose, D-fructose, D-sorbose, and D-tagatose.

In certain embodiments, the carbohydrate is a source of fructose, such as crude fructose, purified fructose, a fructose-containing biomass, corn syrup, sucrose, and polyfructanes.

In certain embodiments, the carbohydrate source is fructose.

In certain embodiments, the carbohydrate source is glucose.

In certain embodiments, the carbohydrate source is sucrose.

The carbohydrate can be present in the alcoholic solvent in any concentration. In certain embodiments, substantially all of the carbohydrate is dissolved in the alcoholic solvent at the temperature the reaction is conducted. In certain embodiments, the carbohydrate is partially dissolved by the alcoholic solvent at the temperature the reaction is conducted. In such instances, the carbohydrate can slowly dissolve in solution as solubilized carbohydrate reacts with the Brønsted acid to form the desired product. In this way, most or all of the carbohydrate starting material can be solubilized and reacted over the course of the reaction.

In certain embodiments the carbohydrate is present in the alcoholic solvent in a concentration of about 0.01 M to about 4 M, from about 0.01 M to about 3 M, from about 0.01 M to about 2 M, from about 0.01 M to about 1 M, or from about 0.3 M to about 1 M. In certain embodiments the carbohydrate is present in the alcoholic solvent in a concentration of at least about 0.01 M, at least about 0.05 M, at least about 0.1 M, at least about 0.2 M, at least about 0.3 M, or at least about 0.4 M. In certain embodiments, the carbohydrate is present in alcoholic solvent at a concentration of at least 0.4 M.

The Brønsted acid can be any protic acid capable of catalyzing the dehydration of a carbohydrate, such as fructose or glucose, to form 5-hydroxymethylfurfural. Such protic acids generally have a pKa of about −10 to about 5 (measured in water). In certain embodiments, the protic acid has a pKa of about −10 to about 4, about −10 to about 3, about −10 to about 2, about −10 to about 2, about −9 to about 2, or about −8 to about 2 (measured in water).

In certain embodiments, the Brønted acid is an inorganic acid selected from the group consisting of H₂SO₄, HSO₄—, H₂SO₃, H₃PO₄, H₂PO₄⁻, HPO₄²⁻, HNO₃, H₂CrO₄, HClO₄, HCl, HBr, and HI. In certain embodiments the Brønsted acid is an inorganic acid selected from the group consisting of H₂SO₄—, H₃PO₄, and HCl.

In certain embodiments, the Brønsted acid is an organic acid selected from the group consisting of carboxylic acids, organic sulfonic acids, organic sulfinic acids, and organic phosphonic acids. The organic group can be selected from alkyl, aryl, haloalkyl, haloalkyl, substituted aryl, and substituted alkyl groups.

In certain embodiments, the Brønsted acid is selected from the group consisting of a hydrogen halide, sulfuric acid, bisulfate salts, alkyl sulfonic acids, aryl sulfonic acids, phosphoric acid, dihydrogen phosphate salts, hydrogen phosphate salts, alkyl phosphoric acids, aryl phosphoric acids, phosphonic acid, and hydrogen phosphite salts.

In certain embodiments, the Brønsted acid is a hydrochloric acid, an alkyl sulfonic acid, an aryl sulfonic acid, or an aryl sulfonic acid resin.

In certain embodiments, the Brønsted acid is hydrochloric acid or an aryl sulfonic acid (exemplified by the commercially available Amberlyst™ resins).

The Brønsted acid catalyzes the formation of 5-hydroxymethylfurfural and can be present in any amount. The Brønsted acid can be delivered neat or in a solvent. When the Brønsted acid is added to the alcoholic solvent as a solution, any solvent can be used for the Brønsted acid, including water, alcohols, such as iso-propanol and tert-butanol; esters, such as ethyl acetate; ethers, such as diethylether, tert-butyl ether, tetrahydrofuran, and 1,4-dioxane; aromatic solvents, such as benzene, toluene, xylenes, and chlorobenzene; chloroalkanes, such as dichloromethane, chloroform, and carbon tetrachloride; and combinations thereof. In certain embodiments, the Brønsted acid is introduced as a solution in water, alcohols, and combinations thereof.

When the Brønsted acid is added, as a solution in a solvent, the concentration of the Brønsted acid can be about 0.01 M to about 16 M. In certain embodiments, the concentration of the Brønsted acid is about 1′M to about 12 M. In certain embodiments, the concentration of the Brønsted acid is about 6 M to about 12 M. In the examples below, the Brønsted acid is HCl and it is delivered to the alcoholic solvent as a 12 M solution in water.

In certain embodiments, the Brønsted acid is added to the alcoholic solvent neat. In instances where the Brønsted acid is a gas, such, as HCl, the Brønsted acid can be added to the alcoholic solvent by bubbling the gaseous Brønsted acid into the alcoholic solvent until the desired concentration of Brønsted acid is achieved in the alcoholic solvent. In instances where the Brønsted acid is a solid or a liquid, the Brønsted acid can be added directly to the alcoholic solvent. In the examples below, the Amberlyst 15 resin is added directly to the alcoholic solvent.

In some instances, addition of the Brønsted acid to the alcoholic solvent can produce an exothermic reaction. In such instances, the Brønsted acid can be added in a manner to minimize the exothermic reaction, for example adding the Brønsted acid slowly, adding the Brønsted acid in portions, and/or adding the Brønsted acid at a′ reduced temperature, e.g., at 23° C., 0° C., or below 0° C.

The rate at which 5-hydroxymethylfurfural is produced in the reaction can be increased by increasing the concentration of Brønsted acid in the alcoholic solvent. The Brønsted acid can be present in a molar ratio from between about 1:99 to about 2:1 relative to the carbohydrate. In certain embodiments, the Brønsted acid is present in a molar ratio from between about 1:99 to about 1:4 relative to the carbohydrate. In certain embodiments, the Brønsted acid is present in a molar ratio from between about 1:49 to about 1:9 relative to the carbohydrate. In certain embodiments, the Brønsted acid is present in a molar ratio from between about 1:19 to about 1:9 relative to the carbohydrate. In the examples below, the Brønsted, acid is present in catalytic amounts, e.g., 10 mol % relative to the carbohydrate.

The alcoholic solvent can comprises any alcohol that is a liquid between the temperatures of 20° C. and 200° C. The alcoholic solvent can comprise a sterically hindered alcohol, such as a secondary alcohol, tertiary alcohol, and combinations thereof.

In certain embodiments, the alcoholic solvent, comprises a secondary or tertiary alcohol of Formula 1

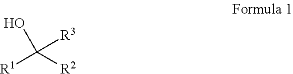

wherein R¹ and R² independently for each occurrence is selected from the group consisting of alkyl, heteroaklyl, alkenyl, alkynyl, cycloalkyl, cycloalkenyl, heterocycloalkyl, heterocycloalkenyl, aryl, aralkyl, heteroaryl, heteroaralkyl, haloalkyl, ether, and ester; and R³ is hydrogen, alkyl, heteroalkyl, alkenyl, alkynyl, cycloalkyl, cycloalkenyl, heterocycloalkyl, heterocycloalkenyl, aryl, aralkyl, heteroaryl, heteroaralkyl, haloalkyl, ether, or ester.

Any secondary alcohol can be used in connection with the processes disclosed herein. Exemplary non-limiting examples of secondary alcohols useful as alcoholic solvents in the present process include iso-propanol, iso-butanol, 2-pentanol, and 3-methyl-2-butanol.

Any tertiary alcohol can be used in connection with the processes disclosed herein. Exemplary non limiting examples of a tertiary alcohol useful as an alcoholic solvent in the present process is tert-butanol.

In certain embodiments, the alcoholic solvent is selected from iso-propanol, tert-butanol, iso-butanol, 2-pentanol, and 3-methyl-2-butanol.

In certain embodiments, the alcoholic solvent is selected from iso-propanol or tert-butanol.

In certain embodiments, the alcohol is an optionally substituted aryl alcohol. In certain embodiments, the alcohol is an aryl alcohol. In certain embodiments, the aryl alcohol is an optionally substituted C6-C14 aryl alcohol. In certain embodiments, the aryl alcohol is an optionally substituted C6-C10 aryl alcohol. In, certain embodiments, the aryl alcohol is selected from optionally substituted phenol or optionally substituted naphthol. In certain embodiments, the aryl alcohol is phenol, 1-naphthol, 2-naphthol, or combinations thereof.

The alcoholic solvent can comprise between about 20% and 100% by volume of a secondary, tertiary alcohol, aryl alcohol, and combinations thereof. Advantageously, as the concentration of the secondary, tertiary alcohol, and/or aryl alcohol increases the amount of side products produced in the reaction decreases and the overall yield of 5-hydroxymethylfurfural increases. In general, when a carbohydrate, such as D-fructose, is dehydrated in the presence of an alcoholic solvent a number of products can be formed. Scheme 1 below depicts four representative products that can result in the dehydration of D-fructose in an alcoholic solvent.

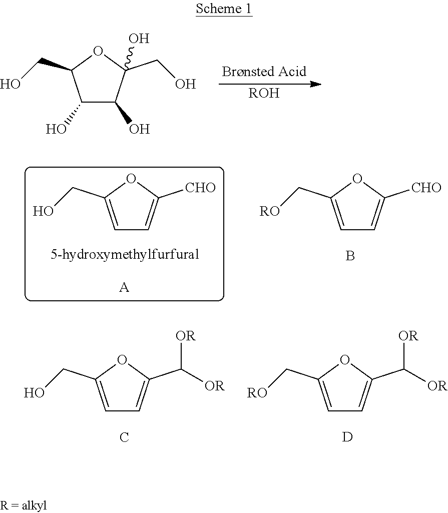

As can be seen in Scheme 1, four products can be formed when D-fructose is reacted with a Brønsted acid in an alcoholic solvent. Reaction products B, C, and D are generated by the reaction of one or more equivalents of the alcoholic solvent with the alcohol or aldehyde functional groups and/or related reaction intermediates as the reaction is allowed to proceed, the desired product A and related intermediates react with additional equivalents of alcohol to form the undesired products, B, C, and D. When less sterically hindered alcohols are used, increasing amounts of the three side-products B, C, and D are also generated and yield of 5-hydroxymethylfurfural is reduced. However, if alcoholic solvents comprising a secondary alcohol, tertiary alcohol, and combinations thereof are employed the desired product A can be formed exclusively.

The aforementioned impurities can also complicate the isolation and purification of 5-hydroxymethylfurfural leading to a further reduction in yield of the desired product and increased cost of production. Surprisingly, when an alcoholic solvent comprising increasing amounts of a secondary or tertiary alcohol is used, 5-hydroxymethylfurfural can be produced exclusively with little or no side products B, C, and D generated. In this regard, the disclosed process provides a greatly enhanced means for efficiently producing 5-hydroxymethylfurfural from carbohydrates.

In certain embodiments, substantially no 5-alkoxymethylfurfural, 5-hydroxymethylfurfural acetal, or 5-alkoxymethylfurfural acetal is present in the reaction product containing 5-hydroxymethylfurfural produced by the processes described herein.

In the examples below, 5-hydroxymethylfurfural is produced in up to an 85% yield with little or no formation of side products B, C, and D. The crude 5-hydroxymethylfurfural produced by the process disclosed herein can be purified by simply filtering off the solids present in the reaction product containing 5-hydroxymethylfurfural and distilling the filtrate.

In contrast, prior art methods for producing 5-hydroxymethylfurfural require labor intensive liquid-liquid extractions to isolate the highly water soluble 5-hydroxymethylfurfural from aqueous or ionic liquid solutions followed by distillation, which can be further complicated by the presence of co-distillate side-products B, C, and D.

The process provided herein employs cheap and environmentally friendly alcoholic solvents, which reduces the cost and environmental impact of producing 5-hydroxymethylfurfural and simplifies purification of the desired product.

In certain embodiments, the alcoholic solvent comprises at least 20%, at least 30%, at least 40%, at least 50%, at least 60%, at least 70%, at least 80%, at least 90%, at least 95%, or at least 98% by volume of a secondary, tertiary alcohol, and/or aryl alcohol.

Advantageously, increasing the amount of secondary or tertiary alcohol, and/or aryl alcohol decreases the amount of side-products and increases the yield of 5-hydroxymethylfurfural. In the examples below, the amount of the secondary or tertiary alcohol is varied between 100% and 0% by volume of the alcoholic solvent. The yield and purity of 5-hydroxymethylfurfural increases as the percent volume of the secondary or tertiary alcohol increases in the alcoholic solvent.

In certain embodiments, the alcoholic solvent comprises at least 80% by volume of the alcohol.

In certain embodiments, the alcoholic comprises at least 90% by volume of the alcohol.

In certain embodiments, alcoholic solvent comprises at least 80% by volume of a secondary alcohol, a tertiary alcohol, and combinations thereof.

In certain embodiments, alcoholic solvent comprises at least 90% by volume of a secondary alcohol, a tertiary alcohol, and combinations thereof.

In certain embodiments, alcoholic solvent comprises at least 80% by volume of a secondary alcohol, a tertiary alcohol, an aryl alcohol, and combinations thereof.

In certain embodiments, alcoholic solvent comprises at least 90% by volume of a secondary alcohol, a tertiary alcohol, an aryl alcohol, and combinations thereof.

The alcoholic solvent may also comprise other solvents, such as water, primary alcohols, ethers, and combinations thereof.

Suitable primary alcohols include methanol, ethanol, propanol, butanol, pentanol, and the like. The primary alcohol can comprise between 0% to about 50% of the alcoholic solvent. In certain embodiments, the primary alcohol is present between 0% and about 20%, between 0% and about 10%, or between 0% and about 5% by volume of the alcoholic solvent.

Suitable ethers include diethyl ether, tetrahydrofuran, 1,4 dioxane, tert-butyl methyl ether, and the like. The ether can comprise between 0% to about 50% of the alcoholic solvent. In certain embodiments, the ether is present between 0% and about 20%, between 0% and about 10%, or between 0% and about 5% by volume of the alcoholic solvent.

In certain embodiments, the alcoholic solvent comprises water. The alcoholic solvent can comprise up to 20% water by volume. Advantageously, the presence of small amounts of water in the reaction can increase the yield of the desired 5-hydroxymethylfurfural product. In such instances, the presence of between 1-8% water by volume in the reaction mixture can improve the yield of 5-hydroxymethylfurfural. The water can be added to an anhydrous alcoholic solvent or a wet alcoholic solvent can be used. As such, non-anhydrous or wet alcoholic solvents can be used in connection with the process disclosed herein. The use of wet alcoholic solvents can further decrease the cost of the process disclosed herein and improves the environmental impact of the process.

In certain embodiments, water can be added to the alcoholic solvent as a solution of the Brønsted acid in water.

In certain embodiments, less than 10% water by volume is present in the step of contacting the carbohydrate and the Brønsted acid in the alcoholic solvent.

The carbohydrate and the Brønsted acid can be contacted in the alcoholic solvent at any temperature. In general, the rate of the reaction between the carbohydrate and Brønsted acid increases as the temperature increases. In certain embodiments, the reaction is conducted at a temperature at or above 20° C. In certain embodiments, the reaction is conducted at 0-20° C. below the boiling point of the alcoholic solvent. In certain embodiments, the reaction is conducted at 0-10° C. below the boiling point of the alcoholic solvent. In certain embodiments, the reaction is conducted at the boiling point of the alcoholic solvent. In certain embodiments, the reaction is conducted at a temperature between 20° C. and 200° C. In certain embodiments, the reaction is conducted at a temperature between 60° C. and 100°. C. In certain embodiments, the reaction is conducted at a temperature between 60° C. and 90° C.

In general, the reaction is allowed to proceed until the most or all of the starting material in consumed. In certain instances, it may be desirable to stop the reaction prior to the consumption of all of the starting material to minimize the amount of side-products formed. In other instances, it may be desired to allow the reaction to continue after all of the starting material is consumed. In certain embodiments, the reaction is allowed to react for about 0.5 to 8 hours. In certain embodiments the reaction is allowed to react for about 1 hour to about 7, about 1 hour to about 6 hours, about 1 hour to about 5 hours, or about 1 hour to about 4 hours. In certain embodiments, the reaction is allowed to react for about 1, about 2, about 3, about or 4 hours. In general, the amount of the desired product increases as the reaction is allowed to continue. However, in certain instances, upon prolonged reaction, the amount of the desired product can decrease as it reacts with the alcoholic solvent producing one of more side-products.

Once the reaction has reached the desired product distribution, it can be stopped or substantially slowed by cooling the reaction, quenching the reaction, e.g., by addition of a base, and/or adding a solvent to dilute the reaction components.

Once the reaction has reached the desired product distribution, the reaction product containing 5-hydroxymethylfurfural can be purified.

The reaction product containing 5-hydroxymethylfurfural can be purified using any known method. Such methods include filtration, distillation, chromatography, liquid-liquid extractions, liquid-solid extractions, or crystallization.

In certain embodiments, the Brønsted acid is removed from the reaction product containing 5-hydroxymethylfurfural by evaporation, filtration, or quenching with an aqueous base. In certain embodiments, the Brønsted acid is removed from the reaction product containing 5-hydroxymethylfurfural by evaporation.

In certain embodiments, the alcoholic solvent is removed from the reaction product containing 5-hydroxymethylfurfural by evaporation under atmospheric pressure or under a partial vacuum.

In certain embodiments, the process for preparing 5-hydroxymethylfurfural further comprises the steps of filtering the reaction product containing 5-hydroxymethylfurfural thereby forming a filtrate, collecting the filtrate and removing the alcoholic solvent from the filtrate by evaporation thereby forming crude 5-hydroxymethylfurfural.

In certain embodiments, the processes for preparing 5-hydroxymethylfurfural comprises the steps of contacting fructose and hydrochloric acid in an alcoholic solvent comprising at least 80% by volume of an alcohol selected from the group consisting of iso-propanol and tert-butanol, and combinations thereof at a temperature of about 60° C. to about 140° C. for about 1 hour to about 3 hours thereby forming a reaction product containing 5-hydroxymethylfurfural.

In one of the examples below, the reaction product containing 5-hydroxymethylfurfural is purified by first filtering the reaction product containing 5-hydroxymethylfurfural and distilling the filtrate to isolate crude 5-hydroxymethylfurfural.

The crude 5-hydroxymethylfurfural produced by the present process can be about 40%, about 50%, about 60%, about 70%, about 80%, about 90% pure, about 95% pure, about 97% pure, about 98% pure, or about 99% pure by mass. In certain embodiments, the crude 5-hydroxymethylfurfural produced by the present process is about 90% to about 99% pure, about 92% to about 99% pure, about 94% to about 99% pure, or about 95% to about 99% pure by mass.

In one of the examples below, the reaction product containing 5-hydroxymethylfurfural is purified by first quenching the reaction product with a basic aqueous solution to neutralize the Brønsted acid. The resulting mixture is then concentrated to remove the alcoholic solvent. Water is then added and the resulting mixture is extracted with an organic solvent. The organic solvent is then evaporated under reduced pressure to afford crude 5-hydroxymethylfurfural.

The crude 5-hydroxymethylfurfural can optionally be further purified using any purification method known in the art. Such methods include filtration, distillation, chromatography, liquid-liquid extractions, liquid-solid extractions, or crystallization

Distillation can be conducted at atmospheric pressure or under reduced pressure. Generally, the temperature at which the reaction components can be distilled at is decreased under reduced pressure.

5-hydroxymethylfurfural boils at about 114-115° C. at 1 kPa. In order to simplify the purification of the desired product by distillation the alcoholic, solvents employed should ideally not have similar boiling points to 5-hydroxymethylfurfural.

In certain embodiments, the crude 5-hydroxymethylfurfural is purified by distillation at atmospheric pressure or under reduced pressure. Simple distillation or, fractional distillation can be used to purify the crude 5-hydroxymethylfurfural to yield purified 5-hydroxymethylfurfural.

Purified 5-hydroxymethylfurfural produced by the present process can be about 80%, about 85%, about 90%, about 95%, about 97%, or about 99%, or >99% pure by mass.

In certain instances, where the reaction is conducted at an elevated temperature, the alcoholic solvent and optionally the Brønsted acid can be removed by evaporation directly from the reaction vessel at the same temperature that the reaction is conducted or an elevated temperature. Evaporation of the alcoholic solvent and optionally the Brønsted acid can be conducted at atmospheric pressure or reduced pressure.

In certain instances, where the reaction is conducted at an elevated temperature, the alcoholic solvent and optionally, the Brønsted acid can be removed by evaporation directly from the reaction vessel at a temperature lower than the reaction is conducted. In such instances, the reaction temperature is first reduced to the desired temperature and the alcoholic solvent and optionally the Brønsted acid are then removed by evaporation by evaporation at atmospheric or reduced pressure.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1(a) is a graph comparing the relationship between the 5-HMF yield and reaction time for condensation reactions performed at 100° C. and 120° C. respectively.

FIG. 1(b) is a graph showing the relationship between the amount of water present and the yield of 5-HMF for a condensation reaction performed at 120° C., in the presence of ispropanol and HCl catalyst, for 2 hours.

FIG. 2 is a chart comparing the 5-HMF yields for five different reaction systems under a reaction temperature of 100° C.

FIG. 3 is a graph showing the yield of 5-HMF according to a scale-up manufacture protocol described in Example 8.

### EXAMPLES

Non-limiting examples of the invention will be further described in greater detail by reference to specific Examples, which should not be construed as in any way limiting the scope of the invention.

### Example 1

Example 1 compares the effects of four different alcoholic solvents on the yield of HMF, converted from D-fructose in the presence of a Brønsted acid (HCl in this example) under an elevated temperature. Four reaction systems were prepared by mixing 0.45 grams of fructose with mol % HCl and 5 mL of a variable alcoholic solvent comprising methanol (System 1), ethanol (System 2), iso-propanol (System 3) or tert-butanol (System 4). The detailed protocol is discussed below.

### Protocol

To a flame-dried 15 mL sealed tubes equipped with stirrer bars, fructose (0.45 g, 2.5 mmol), alcohol (5 mL) and hydrochloric acid (10 M, 0.02 ml) were added. The sealed tube was heated in oil bath at 100° C. with stirring.

The reaction was stopped after a desired reaction time by cooling down the tube in an ice/water bath and adding sodium hydroxide (6.25 M, 0.04 mL) to neutralize the catalyst. Solvents in the reaction mixtures were removed by vacuum. 1 mL of distilled water was then added to the residue and a product was extracted by 10 mL of ethyl acetate. The organic layer was collected and evaporated to obtain the crude product to which mesitylene (0.1 g, 0.83 mmol) was added as internal standard.

In this Example, eight different reactions were performed for each reaction system wherein the reaction mixtures are stirred at 80° C. for a duration ranging from 1 hour to 8 hours (Entries 1 to 8). At the end of each reaction, the percentage yield of the 5-HMF (A) and other possibly occurring intermediates B, C and D are analyzed via NMR. The test conditions and results are shown in Table 1 below.

For the methanol system 1, after 8 hrs of reaction, NMR analysis showed that the products A-D were formed in a A:B:C:D ratio of 1:3:4:17 (Table 1, Entry 8). The formation of intermediate products B, C, D is not surprising because the reaction conditions are also suitable for acetalisation and ether formation. However, the total furfural product yield was only 25% for the methanol system. In comparison, for the ethanol system 2, only 5-HMF (A) and intermediate product (B) were produced with 24% and 14% yield respectively. Notably, for the iso-propanol and t-butanol reaction systems, 5-HMF was produced as the sole furfural product with respective yields of 67% and 61%.

Without being bound by theory, it is postulated that this high selectivity for 5-HMF in Systems 3 and 4 could be attributed to the bulkiness of the structures of iso-propanol and t-butanol, which sterically hindered the formation of the intermediates B-D. From these results, it can be demonstrated that both iso-propanol and tert-butanol (secondary and tertiary alcohol respectively) act as suitable solvents for fructose dehydration to HMF to provide high yields (>40% to 60%) and complete or near complete selectivity of 5-HMF (≈100%). Additionally, time dependent analysis indicates that the dehydration reaction generally proceeded faster in the first 4 hours and then slowly proceeded to completion (See also Table 1).

TABLE 1

| | | System 1 | | System 2 | | System 3 | | System 4 | |
|---|---|---|---|---|---|---|---|---|---|
| | | (Methanol) | | (Ethanol) | | (Iso-propanol) | | (Tert-butanol) | |
| | Time | Yield | | Yield | | Yield | | Yield | |
| Entry | (h) | (%) | A:B:C:D | (%) | A:B:C:D | (%) | A:B:C:D | (%) | A:B:C:D |
| 1 | 1 | 7 | 0.7:0.2:4:2 | 14 | 12:2:0:0 | 30 | 30:0:0:0 | 43 | 43:0:0:0 |
| 2 | 2 | 11 | 1:1:4:5 | 22 | 17:5:0:0 | 39 | 39:0:0:0 | 53 | 53:0:0:0 |
| 3 | 3 | 14 | 1:1:4:8 | 28 | 21:7:0:0 | 44 | 44:0:0:0 | 55 | 55:0:0:0 |
| 4 | 4 | 19 | 1:2:5:11 | 29 | 21:8:0:0 | 50 | 50:0:0:0 | 60 | 60:0:0:0 |
| 5 | 5 | 20 | 1:2:5:12 | 33 | 23:10:0:0 | 56 | 56:0:0:0 | 60 | 60:0:0:0 |
| 6 | 6 | 23 | 2:3:4:14 | 35 | 23:12:0:0 | 59 | 59:0:0:0 | 61 | 61:0:0:0 |
| 7 | 7 | 23 | 1:3:4:15 | 37 | 24:13:0:0 | 63 | 63:0:0:0 | 62 | 62:0:0:0 |
| 8 | 8 | 25 | 1:3:4:17 | 38 | 24:14:0:0 | 67 | 67:0:0:0 | 61 | 61:0:0:0 |
| 9* | 4 | 50 | 8:10:11:12 | 57 | 15:43:0:0 | 61 | 21:40:0:0 | 59 | 29:30:0:0 |

### Example 2

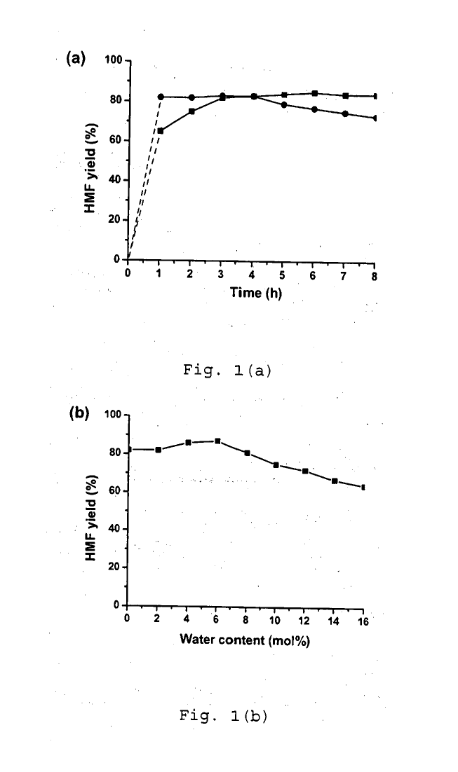

The experimental protocol for Example 1 was followed except that only isopropanol is being used as the alcoholic solvent in this Example. Additionally, the reactions were performed at different temperatures (100° C. and 120° C. respectively) to investigate the effect of temperature on the rate/extent of reaction. The experimental results are provided in FIG. 1.

The inventors found that reactions quickly reach more than 82% yield in less than 1 hour at 120° C. but yield, slowly decreased after 4 hours, which may due to the decomposition/oligomerization of 5-HMF (See FIG. 1 (a)). At 100° C., it took about 3 hours to reach 82% yield and about 5 to 6 hours to reach 85% yield (FIG. 1(a)).

### Example 3

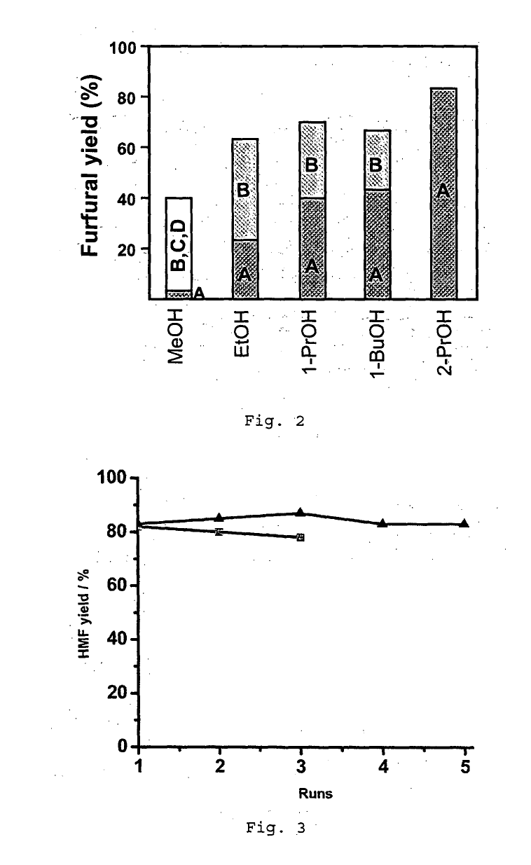

Following the optimized reaction conditions obtained from Example 2 (100° C., 0.45 grams fructose, and 10 mol % HCl, reaction time of 4 hours), other alcohols were again studied as solvents for fructose dehydration. The results are shown in FIG. 2. Referring to chart of FIG. 2, it can be seen that ethanol, 1-propanol and 1-butanol respectively provided about 60%, 73% and 68% yields of a mixture of A (5-HMF) and B, while isopropanol (2-propanol) gave an 83% yield of solely 5-HMF (See FIG. 2). The apparent selectivity towards 5-HMF and superior furfural yield afforded by the use of an alcoholic solvent comprising a secondary alcohol is again evident in this example.

### Example 4

It is expected that trace amounts of water will inevitably be generated during the condensation reaction. Accordingly, the effect of the presence of water is investigated in this Example. The inventors found that the disclosed reaction system does not require water-free conditions. Even with a minor amount of water present (about 6 mol %) in the reaction system, the yield of HMF actually increased to 87%. However, more than 10 mol % of water will result in a decrease of 5-HMF yield (See FIG. 1(b)). Under optimized reaction conditions, up to 87% of 5-HMF yield and 99% of conversion were achieved.

### Example 5

Example 5 investigates the yield of the fructose condensation reaction when a primary alcohol (such as methanol) is used in combination with a secondary alcohol (such as isopropanol) as the alcoholic solvent. Five alcoholic solvent systems were prepared with varying proportions of methanol:isopropanol. System 1 comprised solely methanol solvent and System 5 comprised solely isopropanol solvent. The reaction conditions are as per described in Example 1 (i.e., 0.45 g fructose, 5 mL alcohol, 10 mol % HCl and 80° C. reaction temperature). The proportions of alcohol in each Sample and its accompanying yields are tabulated in Table 2 below.

TABLE 2

| System | CH₃OH:isopropanol | Yield(%) | A:B:C:D |
|---|---|---|---|
| 1 | 10:0 | 25 | 1:3:4:17 |
| 2 | 8:2 | 29 | 3:5:6:15 |
| 3 | 5:5 | 40 | 15:7:9:9 |
| 4 | 2:8 | 48 | 44:4:0:0 |
| 5 | 0:10 | 65 | 65:0:0:0 |

From these results, it can be seen that a mixture of methanol and, iso-propanol did not benefit the reaction yield or the selectivity towards 5-HMF.

### Example 6

In this example, an aryl sulfonic acid polymeric resin (commercially available as Amberlyst 15™ from Rohm Haas) is evaluated as a catalyst for the fructose dehydration reaction in various alcoholic solvents. Initial reactions were tested with methanol, ethanol, iso-propanol and t-butanol and the results are as, shown in Table 1, Entry 9. In particular, reaction in methanol gave about 50% yield of a mixture comprising all of A, B, C and D. Reactions in ethanol, iso-propanol and t-butanol gave around 60% yield of mixture of A and B, whereas it is further noted that reaction in bulky alcohol is more selective towards HMF.

The following protocol for, a fructose dehydration reaction using the Amberlyst rein will be adopted for this Example. To a flame-dried 15 mL sealed tubes equipped with stirrer bars, fructose (0.45 g, 2.5 mmol), iso-propanol (5 mL) and Amberlyst 15 (106 mg, 20 mol %) were added. The sealed tube was heated in an oil bath at 120° C. with stirring. The reaction was stopped after 4 hours, by cooling down the tube in an ice/water bath. The catalyst is removed by filtration and then washed with 5 ml of methanol. The methanol was then combined with the filtrate. The solvents in the filtrates were removed. 1 mL of distilled water was then added to the residue and the product was extracted by 10 mL of ethyl acetate. The organic layer was collected and evaporated to obtain the crude product to which mesitylene (0.1 g, 0.83 mmol) was added as internal standard. The compositions of the samples were then analyzed by NMR. The washed catalysts were dried under vacuum at 100° C. for 1 hour and used directly for the next batch of reaction.

Three reaction runs were performed (a First run, Recycle 1 and Recycle 2). For each run, a total of 8 reactions with reaction times from 1 hour to 8 hours (Entries 1 to 8) were carried out. The yields and product ratio of the reactions for each run and for each reaction time are tabulated in Table 3 below.

Referring to the first run, it can be seen that total furfural yield reached 40% in first hour at 120° C. and increased to maximum 60% after about 4 hours. At four hours, the ratio of products A:B:C:D is 24:28:5:3 (See Entry 4). It can also be seen that the amount of 5-HMF (A) started to decline after 4 hours, even though the total yield of furfural compounds remained fairly constant at about 60%.

For the second run with recycled catalyst, slightly improved results (62% total furfural yield) were achieved at 4 hrs with the products having a A:B:C:D ratio of 39:22:1:0. It can be seen that more 5-HMF was produced in the second run relative to the other intermediates B and C when using the recycled catalyst. This change may indicate that the acidity of recycled catalyst was decreased and which favored selectivity towards 5-HMF (A).

For the third run (second recycle), about 57% of total furfural yield was achieved at 4 hours with a product ratio A:B:C:D of 45:12:0:0. It can be seen that the selectivity of the reaction towards 5-HMF (A) appears to improve with recycled catalysts. This suggests that the Amberlyst resin catalyst may facilitate more selectivity towards 5-HMF (A) while maintaining a fairly, constant total product yield under less acidic conditions.

TABLE 3

| | Time | First Run | | Recycle 1 | | Recycle 2 | |
|---|---|---|---|---|---|---|---|
| Sample | (h) | Yield (%) | A:B:C:D | Yield (%) | A:B:C:D | Yield (%) | A:B:C:D |
| 1 | 1 | 40 | 31:9:0:0 | 46 | 35:11:0:0 | 50 | 27:7:12:5 |
| 2 | 2 | 56 | 39:17:0:0 | 53 | 37:16:0:0 | 57 | 30:12:10:5 |
| 3 | 3 | 57 | 23:23:5:6 | 60 | 40:20:0:0 | 57 | 35:10:8:4 |
| 4 | 4 | 60 | 24:28:5:3 | 62 | 39:22:1:0 | 57 | 45:12:0:0 |
| 5 | 5 | 59 | 14:31:7:7 | 60 | 30:22:4:4 | 52 | 34:8:7:3 |
| 6 | 6 | 58 | 15:34:4:5 | 59 | 29:22:4:4 | 53 | 27:7:15:4 |
| 7 | 7 | 57 | 11:35:4:7 | 58 | 28:21:4:4 | 45 | 38:7:0:0 |
| 8 | 8 | 57 | 12:38:3:5 | 58 | 28:21:4:5 | 47 | 30:4:10:3 |

### Example 7

Example 7 describes a dehydration reaction of glucose in iso-propanol. An initial test carried out with a NHC—Cr(II) (1,3-bis(2,6-diisopropylphenyl)imidazolylidene chromium (II)]) catalyst provided 34% total furfural yield from glucose in iso-propanol. The reaction protocol is as described below.

To a flame-dried 15 ml sealed tube equipped with stirrer bar, glucose (0.45 g, 2.5 mmol), iso-propanol (5 mL) and a NHC—Cr(II) catalyst (0.21 g, 0.5 mmol) were added. The reaction was heated in an oil bath at 120° C. with stirring and stopped after 4 hours by cooling the tubes in an ice/water bath.

Solvents in the reaction mixture were removed. 1 mL of distilled water was added to the residue and the product was extracted with about 10 mL of ethyl acetate. The organic layer was collected and evaporated to obtain the crude product to which mesitylene (0.1 g, 0.83 mmol) was added as internal standard. The composition of the sample was then analyzed by NMR.

### Example 8

This example provides an alternative protocol for the production of HMF in a secondary/tertiary alcohol solvent reaction system, which can be used for scale-up HMF industrial production. In particular, an exemplary scale up protocol can be as follows: To a flame-dried 150 mL flask equipped with stirrer bars, fructose (4.5 g, 25 mmol), isopropanol alcohol solvent (50 mL) and hydrochloric acid (5 M, 0.2 mL) are added. The reaction flask is heated in an oil bath at 120° C. with constant stirring. The reaction is stopped at 4 hours. The reaction mixture is filtered to remove insoluble humin by-product. Solvent in the reaction mixtures are then distilled to obtain a crude HMF product. The solvent can be recycled directly for use in the next reaction run. The conversion results can be seen on FIG. 3.

In this Example, evaporated solvent was directly used for the subsequent reaction runs. An additional amount of HCl (2 mol %) is also added to each subsequent reaction run. As can be seen, the total furfural yield obtained ranges between 78% to about 90%, indicating that good conversion yield can also be achieved based on the disclosed scale-up protocol.

### APPLICATIONS

The present disclosure provides an alcohol-mediated reaction process for production of HMF from sugars, wherein the alcohol is at least one of a secondary alcohol, a tertiary alcohol, an aryl alcohol or a mixture thereof. The disclosed process can achieve up to 87% of 5-HMF yield from fructose, with iso-propanol as solvent and HCl or solid acid (Amberlyst 15) as catalyst under mild conditions. The disclosed process is capable of providing complete selectivity of HMF over other possible alkoxylated side-products.

According to the disclosed process, the solvent and catalyst can be easily removed via evaporation or simple distillation and which can be further recycled and reused. Hence, the disclosed process avoids using large amounts of organic solvent and has limited adverse impact on the environment. The present application further discloses a process for HMF production, which can be readily scaled up for industrial scale production.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A process for preparing 5-hydroxymethylfurfural comprising the step of contacting a carbohydrate and a Brønsted acid in an alcoholic solvent having at least 80% by volume of an alcohol selected from the group consisting of secondary alcohols, tertiary alcohols, aryl alcohols and combinations thereof under conditions to dehydrate the carbohydrate thereby forming a reaction product containing 5-hydroxymethylfurfural.

2. The process of claim 1, wherein the alcoholic solvent has at least 90% by volume of said alcohol.

3. The process of claim 1, wherein the alcoholic solvent has at least 98% by volume of said alcohol.

4. The process of claim 1, wherein the alcohol is selected from the group consisting of secondary alcohols, tertiary alcohols, and combinations thereof.

5. The process of claim 1, wherein the carbohydrate is a source of fructose.

6. The process of claim 5, wherein the source of fructose is crude fructose, purified fructose, a fructose-containing biomass, corn syrup, sucrose, and polyfructanes.

7. The process of claim 1, wherein the conditions to dehydrate the carbohydrate comprise contacting the carbohydrate and the Brønsted acid in the alcoholic solvent at a temperature above 23° C. temperature.

8. The process of claim 7, wherein the temperature is about 60° C. to about 140° C.

9. The process of claim 1, wherein the Brønsted acid is selected from the group consisting of a hydrogen halide, sulfuric acid, bisulfate salts, alkyl sulfonic acids, aryl sulfonic acids, phosphoric acid, dihydrogen phosphate salts, hydrogen phosphate salts, alkyl phosphoric acids, aryl phosphoric acids, phosphonic acid, and hydrogen phosphite salts.

10. The process of claim 9, wherein the Brønsted acid is hydrochloric acid, an alkyl sulfonic acid, an aryl sulfonic acid, or an aryl sulfonic acid resin.

11. The process of claim 10, wherein the Brønsted acid is present in about 1:99 to about 1:9 molar ratio relative to the carbohydrate.

12. The process of claim 1, wherein the alcoholic solvent is selected from iso-propanol, tert-butanol, iso-butanol, 2-pentanol, and 3-methyl-2-butanol.

13. The process of claim 12, wherein the alcoholic solvent is iso-propanol or tert-butanol.

14. The process of claim 1, wherein the contacting step occurs for about 1 to about 8 hours.

15. The process of claim 1, further comprising the steps of filtering the reaction product containing 5-hydroxymethylfurfural thereby forming a filtrate, collecting the filtrate and removing the alcoholic solvent from the filtrate by evaporation thereby forming crude 5-hydroxymethylfurfural.

16. The process of claim 15, wherein the Brønsted acid is removed from the filtrate by evaporation.

17. The process of claim 15, further comprising the steps of purifying the crude 5-hydroxymethylfurfural using a distillation process thereby forming purified 5-hydroxymethylfurfural.

18. The process of claim 1, wherein less than 10% water by volume is present in the step of contacting the carbohydrate and the Brønsted acid in an alcoholic solvent.

19. The process of claim 1, wherein the processes comprises the steps of contacting fructose and hydrochloric acid in an alcoholic solvent comprising at least 80% by volume of an alcohol selected from the group consisting of iso-propanol and tert-butanol, and combinations thereof at a temperature of about 60° C. to about 140° C. for about 1 hour to about 3 hours thereby forming a reaction product containing 5-hydroxymethylfurfural.

20. The process of claim 1, wherein the carbohydrate is present in the alcoholic solvent at a concentration of at least 0.4 molar.

21. The process of claim 1, wherein substantially no 5-alkoxymethylfurfural, 5-hydroxymethylfurfural acetal, or 5-alkoxymethylfurfural acetal is present in the reaction product containing hydroxymethylfurfural.
